(19) United States
(12) Patent Application Publication
(10) Pub. No.: US 2025/0127735 A1
(43) Pub. Date: Apr. 24, 2025

(54) NOVEL COMPOSITION COMPRISING N-ACETYLCYSTEINE FOR OVERCOMING THE ADVERSE EFFECTS OF A CHEMOTHERAPY

(71) Applicants: PRONUTRI, CARROS (FR); UNIVERSITE COTE D'AZUR, NICE (FR); CENTRE NATIONAL DE LA RECHERCHE SCIENTIFIQUE, PARIS (FR)

(72) Inventors: Laurent COUNILLON, NICE (FR); Amalric VERET, CARROS (FR)

(73) Assignees: PRONUTRI, CARROS (FR); UNIVERSITE COTE D'AZUR, NICE (FR); CENTRE NATIONAL DE LA RECHERCHE SCIENTIFIQUE, PARIS (FR)

(21) Appl. No.: 18/834,282

(22) PCT Filed: Jan. 27, 2023
(86) PCT No.: PCT/EP2023/051988

§ 371 Date: Jul. 30, 2024

(30) Foreign Application Priority Data
Jan. 31, 2022 (FR) 2200809

(51) Int. Cl. A61K 31/19; A61K 9/16; A61K 31/455; A61K 31/51; A61K 31/525; A61K 31/714

(52) U.S. Cl. CPC A61K 31/19; A61K 9/1623; A61K 9/1664; A61K 31/455; A61K 31/51; A61K 31/525; A61K 31/714

(57) ABSTRACT

Compositions for use in the prevention and/or treatment of at least one adverse effect of a chemotherapy in the treatment of cancer are disclosed. The compositions have N-acetylcysteine (NAC) and vitamin B12 and/or a combination of vitamins B1, B2 and B3. Methods of treatment of chemo-induced nociceptive and/or neuropathic pain by administering such compositions are also disclosed.

### TECHNICAL FIELD

The invention relates to a composition comprising N-acetylcysteine (NAC) in combination with other active agent(s) for use thereof in the prevention and/or treatment of at least one adverse effect of a chemotherapy in the treatment of cancer, in particular the prevention and/or treatment of chemo-induced nociceptive and/or neuropathic pain.

### PRIOR ART

N-acetylcysteine, or NAC, is a non-essential cysteine-derived amino acid having an acetyl group attached to the nitrogen atom.

NAC is therefore a thiol that can oxidize, reducing the amount of free radicals. It is also a precursor of glutathione, a well-known antioxidant.

It is known for use as a drug and as a dietary supplement.

For example, NAC is commonly used to treat acetaminophen overdoses. NAC may also be of use in other oxidative stress conditions such as apoptosis, mitochondrial dysfunction, neuroinflammation, and also glutamate and dopamine dysregulation.

In particular, NAC can be used to improve the symptoms of several types of drug-induced neuropathy.

Chemotherapy-induced peripheral neuropathy (CIPN), induced for example by platinum salts, taxanes, vinca alkaloids, bortezomib, thalidomide and eribulin, for example, is a barrier to their use.

This is because these adverse effects, which are usually dose-dependent and dose-limiting, force clinicians to reduce the dose or even to stop treatment prematurely, thereby compromising the patient's chances of recovery. The clinical picture of CIPN includes sensory symptoms, which may or may not be accompanied by motor and/or autonomic symptoms that can be severe and incapacitating. CIPN occurs both acutely (oxaliplatin and paclitaxel) and chronically.

Neuropathies induced by anti-cancer agents may appear immediately, but more often they develop gradually and may persist for several years after treatment has ended, and the clinical symptoms may become irreversible. This suggests that a series of different mechanisms are established, which may range from instant effects to remodeling and ultimately to neuronal damage.

Table 1 below provides a quick overview of the different types of neuropathic effects suffered by patients treated with platinum derivatives or taxanes.

TABLE 1
Side effects of chemotherapies observed clinically

| Anti-cancer | | | Motor | Autonomic | | |
|---|---|---|---|---|---|---|
| agent | Sensory effects | Pain | effects | effects | Reflexes | Recovery |
| Cisplatin | Paresthesia, | Dysesthesia | Normal | Rare | Reduced | |
| | tremors, | | | (orthostatic | | |
| | proprioception, | | | dysregulation) | | |
| | unpredictable | | | | | |
| | heat sensitivity | | | | | |
| Carboplatin | Similar to cisplatin | | | | | |
| Oxaliplatin | | Dysesthesia, | Muscle | Normal | Normal | Recovery |
| (acute | | allodynia to | cramps | | | after a few |
| reaction) | | cold, | | | | days |
| | | mechanical | | | | |
| | | hyperalgesia | | | | |
| Oxaliplatin | Similar to cisplatin | | | | | |
| (chronic) | | | | | | |
| Paclitaxel, | Paresthesia, | Dysesthesia, | Rare | Rare | Reduced | Generally |
| docetaxel | tremors, | painful | (proximal > | (orthostatic | | no recovery |
| | proprioception, | burning | distal | dysregulation) | | and possible |
| | heat and | sensation, | weakness) | | | progression |
| | mechanical | paradoxical | | | | after |
| | sensitivity | heat | | | | treatment is |
| | | sensitivity | | | | stopped |

For short- or medium-term side effects, mechanical, thermal or pain-related stimuli are detected in all cases and transmitted by ion channels; the main known ion channels are listed in table 2 below. Cisplatin has been shown to have an acute effect on some of these channels (Milosavljevic et al., Cancer Res., 2011).

TABLE 2
Examples of channels involved in nociception and mechanosensation

| | |
|---|---|
| Channels | Role |

Threshold potassium channels

| | |
|---|---|
| TREK1/TRAAK | Neuroprotection, pain, suppression |
| TREK2 | Polymodal pain/nociception, sensitivity |
| | to hot and cold, allodynia to cold |
| | associated with oxaliplatin |
| Kv1.1/1.2/1.4 | Peripheral neuropathies |

Excitatory sodium channels

| | |
|---|---|
| ASIC1a/ASIC2a/ASIC3 | Nociception in tissue acidosis and |
| | inflammation |
| Nav1.7/Nav1.8/Nav1.9 | Allodynia and inflammatory pain |

Other excitatory channels

| | |
|---|---|
| P2X2-3/3/P2X4 | Allodynia and inflammatory pain |
| TRPV1/TRPM8/TRPA1 | Heat sensitivity |
| Piezo 2 | Light touch and pain perception |
| Caᵥα2δ-1 | Touch sensitivity |
| (Cav1.2/1.3/Cav2.1-2.3) | |

Numerous preventive and curative treatments have been tested within this indication, that is chemo-induced neuropathies, with varying degrees of success (Thesis by Thibaut Fayolle, “Stratégie thérapeutique des neuropathies périphériques chimio-induites” [Therapeutic strategy for chemo-induced peripheral neuropathies], UNIVERSITE CLERMONT AUVERGNE, 2018).

Examples of compounds used in the prevention and treatment of chemo-induced neuropathies (Emilie Le Rhun, “Caractéristiques cliniques des neuropathies chimio-induites” [Clinical characteristics of chemo-induced neuropathies], CHRU Lille and Unicancer, 2016) include:
from the compounds that do not appear to be of preventive interest: lipoic acid, nimodipine, gabapentin, lamotrigine, acetyl L-carnitine, venlafaxine;
from the compounds exhibiting varying preventive results: amifostine, glutathione, calcium and magnesium infusions, vitamin E;
from the compounds of possible preventive interest: N-acetylcysteine, carbamazepine and oxcarbazepine, Xaliproden (5-hydroxytryptamine 1A agonist), Goshajinkigan, erythropoietin, PFT-μ, omega-3 fatty acid, baclofen-containing topical gel, amitriptyline, nortriptyline and ketamine, vitamin B12/B6 combination;
from the pharmaceutical compounds of possible curative interest: duloxetine;
from the pharmaceutical compounds with a debatable curative effect: gabapentin or pregabalin, venlafaxine, amitriptyline, serotonin and norepinephrine reuptake inhibitors, opioids, local anesthetics, lidocaine infusion, dextromethorphan;
from the compounds with no apparent curative effect: lamotrigine, imipramine.

For this purpose, document WO03045334 is also known, which describes a chemoprotective composition (the chemotherapy agent being, for example, cisplatin) comprising at least two chemoprotectants selected from the group consisting of methionine, N-acetyl-DL-methionine, S-adenosylmethionine, cysteine, homocysteine, cystathione, cysteamine, N-acetylcysteine, glutathione, glutathione ethyl ester, glutathione, glutathione diethyl ester triethyl ester, cysteamine, DiNAC, RibCys, RibCyst, ss-LactCys, a-LactCys, MeliCys, MaltCys, CellCys, OTCA, allopurinol, 1-methylallopurinol, 2-methylallopurinol, 5-methylallopurinol 1,7-methylallopurinol, 7-methylallopurinol, 7-methylallopurinol, 2,5-dimethylallopurinol, 1,7-dimethylallopurinol, 2,7-dimethylallopurinol, 5,7-dimethylallopurinol, 2,5,7-trimethylallopurinol, 1-ethoxycarbonylallopurinol, 1-ethoxycarbonyl-5-methylallopurinol, 2-phenyl-1,2-benzoisoselenazol-3(2H)-one and 6-diSeCD.

Similarly, document WO2007044700 relates to a therapeutic combination comprising two or more protective agents selected from a group consisting of specific methionine protective agents, N-acetylcysteine, carnitine, magnesium ions, lipoic acid, ebselen, glutathione and glutathione ester, administered to treat ototoxicity, nephrotoxicity, neurotoxicity, alopecia, gastrointestinal disorders or reduced survival in a patient undergoing treatment with a chemotherapeutically effective amount of an antitumor platinum compound.

Document WO0076475 is also known, which describes compositions for use thereof in the prevention or treatment of the adverse effects of radiotherapy or chemotherapy, in particular an example explicitly containing 50 ppm cyanocobalamin (vitamin B12) and 500 ppm N-acetylcysteine, and also 200 ppm KCl and 500 ppm green tea extract. However, such a composition appears to have a limited protective effect on vincristine-induced neuropathies.

Document CN113693231 describes a dietary supplement comprising in particular γ-glutamylcysteine and vitamins, in particular B1, B2, B6 and B12, to provide nutrition and combat the toxic effects and side effects caused by radiotherapy and chemotherapy, with a significant effect on damage to the mucous membranes of the digestive tract.

The publication by Lieberman et al. “Poly-MVA for treating non-small-cell lung cancer: A case study of an integrative approach” (04/2006) describes a patient treated with chemotherapy (5FU and mitomycin C) for lung cancer and taking a supplement comprising acetylcysteine, cyanocobalamin, thiamine and riboflavin.

Furthermore, document US20020058628 discloses a method for treating a disease or condition for which the treatment involves promoting the growth of neuronal cell processes and the therapy for which comprises administering an agonist for neuronal cell surface receptors that promote the growth of neuronal processes, for example chemotherapy-induced neuropathy, said method comprising the administration of a therapeutically effective amount of said agonist in combination with a therapeutically effective concentration of one or more antioxidants/free radical scavengers and/or an agent which is able to increase intracellular thiol levels and/or a steroid. A representative intracellular thiol is glutathione, the agent which is able to raise intracellular thiol levels is N-acetylcysteine (NAC), and representative free radical scavenging antioxidants can be selected from the group consisting of vitamin C, vitamin E, analogs thereof and mixtures thereof. A representative analog of vitamin E is trolox.

Finally, it is known that vitamin B12 deficiency can lead to various neurological and behavioral disorders such as ataxia, muscle weakness, incontinence, hypotension, vision disorders, psychotic disorders and mood disorders.

However, the data does not appear sufficient to conclude that any of the potential chemoprotective agents, for instance acetylcysteine, amifostine, calcium and magnesium, diethyldithiocarbamate, glutathione, ORG 2766, oxcarbazepine, retinoic acid or vitamin E, can effectively prevent or limit the neurotoxicity of platinum-based drugs in human patients, as has been determined using objective quantitative measures of neuropathy (Albers J W et al., “Les interventions pour la prévention des lésions nerveuses causées par le cisplatine et d'autres medicaments anti-tumoraux a base de platine” [Interventions for the prevention of nerve damage caused by cisplatin and other platinum-based anti-tumor drugs], Cochrane Database of Systematic Reviews 2014, Issue 3. Art. No.: CD005228. DOI: 10.1002/14651858.CD005228.pub4).

### Technical Problem

In view of the above, one problem addressed by the present invention consists in developing a novel composition comprising N-acetylcysteine (NAC) and having a potentiated effect in the prevention and/or treatment of at least one adverse effect of a chemotherapy in the treatment of cancer.

### Solution Provided

The first subject of the solution to this stated problem is a composition comprising, in a physiologically acceptable medium, N-acetylcysteine (NAC), characterized in that it further comprises vitamin B12 at a concentration of between 0.04 and 0.08% by weight of the total weight of the composition and/or a combination of vitamins B1, B2 and B3, for use thereof in the prevention and/or treatment of chemo-induced nociceptive and/or neuropathic pain.

Surprisingly, from the number of possible combinations of chemoprotective agents, and considering the combinations already envisaged and tested, in particular with N-acetylcysteine (NAC), it is not obvious that a potentiated effect can be obtained with the specific use of N-acetylcysteine (NAC) combined with vitamin B12 and/or a combination of vitamins B1, B2 and B3 for overcoming the adverse effects of chemotherapy.

### Advantages Provided

The Applicant was in particular surprisingly able to develop a composition comprising NAC in combination with other active agent(s) which is particularly effective in the prevention and/or treatment of at least one adverse effect of a chemotherapy in the treatment of cancer, in particular the prevention and/or treatment of chemo-induced nociceptive and/or neuropathic pain.

More particularly, the composition according to the invention makes it possible to reduce the useful doses of NAC while retaining the same effect.

### BRIEF DESCRIPTION OF THE DRAWINGS

The invention and its advantages will be better understood on reading the following description and non-limiting embodiments, shown with reference to the appended drawings wherein:

FIG. 1 shows the effect of taxanes (A and C) and cisplatin (B and D) on expression of the Cavα2δ-1 subunit (A and B) and the Piezo 2 channel (C and D), which are involved in pain transmission and detection of mechanical stimuli, respectively.

FIG. 2 shows the transcription factors targeting genes, the expression of which is altered after treatment with taxanes and/or cisplatin platinum in DRG primary cultures.

FIG. 3 shows the single kinetics of inhibition of the genes Piezo 2, CaVα2δ-1 and ARNT2 after 0, 4, 8 and 16 h of treatment with cisplatin (1.25 μg/ml) in mouse DRGs resulting from primary culture.

FIG. 4 shows the expression of Piezo 2, CaVα2δ-1 and ARNT2 in primary cultured neurons treated with an oxidizing agent (tert-butyl hydroperoxide, TBHP).

FIG. 5 shows the dose-response of NAC on Arnt2, Piezo 2 and CaVα2δ-1 gene expression after 24 h of exposure to 1.25 μg/ml cisplatin.

FIG. 6 shows the dose-response of beta-carotene on Arnt2, Piezo 2 and CaVα2δ-1 gene expression after 24 h of exposure to 1.25 μg/ml cisplatin.

FIG. 7 shows the dose-response of vitamin B12 on Arnt2, Piezo 2 and CaVα2δ-1 gene expression after 24 h of exposure to 1.25 μg/ml cisplatin.

FIG. 8 shows the dose-response of luteolin on Arnt2, Piezo 2 and CaVα2δ-1 gene expression after 24 h of exposure to 1.25 μg/ml cisplatin.

FIG. 9 shows the effect of combinations of NAC and different vitamins on Arnt2, Piezo 2 and CaVα2δ-1 gene expression after 24 h of exposure to 1.25 μg/ml cisplatin.

FIG. 10 shows the effect of combinations of NAC and beta-carotene on Arnt2, Piezo 2 and CaVα2δ-1 gene expression after 24 h of exposure to 1.25 μg/ml cisplatin.

### DESCRIPTION OF EMBODIMENTS

In this description, unless otherwise specified, it is understood that, when an interval is given, it includes the upper and lower bounds of said interval.

The invention relates to a composition comprising, in a physiologically acceptable medium, N-acetylcysteine (NAC) for use thereof in the prevention and/or treatment of at least one adverse effect of a chemotherapy in the treatment of cancer, and more particularly of chemo-induced nociceptive and/or neuropathic pain.

NAC is a non-essential cysteine-derived amino acid having an acetyl group attached to the nitrogen atom.

NAC is therefore a prodrug of the endogenous amino acid L-cysteine, a precursor of glutathione, and is known to possess mucolytic, antioxidant and potentially cytoprotective, anti-cancer and anti-inflammatory activities.

When administered, acetylcysteine exerts its mucolytic activity by reducing disulfide bonds in mucoproteins, causing mucus to liquefy and reducing its viscosity.

Its antioxidant activity is attributed to the ability of GSH to scavenge reactive oxygen species (ROS), thereby preventing ROS-induced cell damage, reducing oxidative stress, protecting cells against the damaging effects of free radicals and preventing apoptosis in these cells.

Moreover, this can inhibit tumor cell proliferation, progression and survival, in sensitive tumor cells that depend on ROS-mediated signaling for their proliferation and malignant behavior.

Under certain circumstances, acetylcysteine is able to induce apoptosis in susceptible cells, including some tumor cells, via the intrinsic mitochondria-dependent pathway but which does not involve stress on the endoplasmic reticulum.

Acetylcysteine also has anti-inflammatory activity via modulation of the nuclear factor-kappa B (NF-κB) pathway and modulation of cytokine synthesis.

The composition according to the invention is characterized in that it further comprises vitamin B12 at a concentration of between 0.04 and 0.08% by weight of the total weight of the composition and/or a combination of vitamins B1, B2 and B3, preferentially vitamin B12 at a concentration of between 0.04 and 0.08% by weight of the total weight of the composition, taken alone or associated with the combination of vitamins B1, B2 and B3.

Vitamin B12, also known as cobalamin, is a highly complex essential water-soluble vitamin containing the mineral cobalt. This vitamin is produced naturally by intestinal microorganisms and is also found in soil and water. It is necessary for DNA synthesis and cellular energy production.

Vitamin B12 has numerous forms, including the cyano, methyl, deoxyadenosyl and hydroxy-cobalamin forms. The cyano form is the most widely used.

Advantageously, the composition according to the invention further comprises a combination of vitamins B1, B2 and B3, in different or equal proportions, preferentially in equal proportions, taken alone or preferentially in combination with vitamin B12.

Vitamin B1, also known as thiamine, is a metabolic precursor of thiamine pyrophosphate (TPP), an essential coenzyme for some decarboxylases. It is essential for the conversion of carbohydrates into energy via the Krebs cycle, and is necessary for the proper functioning of the nervous system and muscles. Indeed, it is essential for the conversion of pyruvate, which is produced by glycolysis and is toxic to the nervous system. It can also inhibit the action of glucose and insulin in the proliferation of arterial smooth muscle cells. Thiamine may also protect against lead toxicity by inhibiting lead-induced lipid peroxidation.

Vitamin B2, or riboflavin, is a vitamin which is necessary for the synthesis of flavin adenine dinucleotide (FAD) and flavin mononucleotide (FMN), two essential cofactors for flavoproteins. These cofactors are of vital importance in normal tissue respiration, pyridoxine activation, conversion of tryptophan to niacin, lipid, carbohydrate and protein metabolism, and glutathione reductase-mediated detoxification.

Riboflavin may also be involved in maintaining the integrity of erythrocytes. It is also essential for healthy skin, nails and hair.

Niacin, also known as nicotinic acid and vitamin B3, is a water-soluble essential B vitamin which, when administered in high doses, is effective in lowering the level of low-density lipoprotein (LDL) cholesterol and raising the level of high-density lipoprotein (HDL) cholesterol, making it uniquely valuable in the treatment of dyslipidemia.

The composition according to the invention is used in the prevention and/or treatment of at least one adverse effect of a chemotherapy in the treatment of cancer, and more particularly of chemo-induced nociceptive and/or neuropathic pain.

Cancer is a disease characterized by the uncontrolled proliferation of cells, associated with eluding regulatory mechanisms that ensure the harmonious development of the organism.

Cancer starts with a normal cell that transforms and becomes abnormal. This transformation may be due to: constant activation of proto-oncogenes, inactivation of tumor suppressor genes, or inactivation of DNA repair genes.

The cancers which are more particularly targeted according to the invention result from solid tumors and are preferentially chosen from testicular, prostate, ovarian, cervical, breast, bladder cancers, ENT, head and neck, esophageal, liver, lung or brain tumors, or neuroblastomas.

There are currently several anti-cancer therapies available: surgery, radiotherapy and chemotherapy.

Chemotherapy is a pharmacological treatment that makes it possible to target signaling pathways or the tumor microenvironment. It is the most widely used treatment for metastasized cancers. It can be combined with other therapies. Chemotherapy makes it possible to target cell division and therefore the cell cycle: some treatment molecules act as DNA intercalators, others affect enzymes that control DNA topology, thereby disrupting DNA replication; some molecules target microtubules, and finally others form adducts between the 2 DNA strands, which are detected as damage to this molecule and thus lead to cell death via various signaling pathways.

However, chemotherapy has certain limitations. Indeed, it also targets normal cells, and is therefore toxic to these cells.

A physiologically acceptable medium denotes a medium that is compatible and suitable for use in contact with human and animal cells, in particular with the skin, mucous membranes and/or skin appendages, without toxicity, irritation, inappropriate allergic response or the like, and proportionate to a reasonable benefit-risk ratio.

A physiologically acceptable medium according to the invention may comprise any excipient which is known and used in the pharmaceuticals and food supplement field and which is compatible with NAC and vitamins B12 and/or B1, B2 and B3 used according to the invention.

As non-limiting examples, mention may be made of solvents, buffers, flavoring agents, binders, chelating agents, surfactants, thickeners, humectants, moisturizers, preservatives, antioxidants, soothing agents, colorants, fragrances and the like, or mixtures thereof.

Naturally, those skilled in the art will take care to choose the potential compound(s) to be added to these compositions in such a way that the advantageous properties intrinsically attached to the present invention are not, or not substantially, adversely affected by the envisaged addition. Their concentration is also chosen so as not to impair the advantageous properties of the compounds used according to the invention.

As examples of excipients, mention may be made of xylitol, shellac, lactose, sorbitol, sucrose, wheat starch, magnesium stearate, lipoic acid, or omega-3-rich oils such as linseed oil, perilla oil, hempseed oil, camelina oil, Inca inchi oil, walnut oil, rapeseed oil, chia oil, sweet briar oil from Chile, sea buckthorn oil.

The composition according to the invention is preferentially used for the prevention and/or treatment of chemo-induced nociceptive and/or neuropathic pain, more preferentially when chemotherapy is induced by platinum derivatives selected from cisplatin, oxiplatin and carboplatin, or by taxanes selected from paclitaxel and docetaxel.

Taxanes and platinum derivatives are used, as first-line treatment or in combination therapy, in over 80% of cancers treated with chemotherapy. They can be used to treat cancers of the ovary, breast, liver, lung, head and neck, testicle, prostate and esophagus.

Taxanes are a class of diterpenes. The main mechanism of action of the class of the taxanes is inhibition of microtubule function. Microtubules are essential for cell division, and taxanes block normal cell division. Taxanes thus act as mitotic spindle poisons. They are also believed to be radio-sensitizing.

This class includes paclitaxel, known under the brand name Taxol. Paclitaxel is a molecule produced by endophytic fungi and is also found in extracts of certain yew species, giving them their high toxicity.

Paclitaxel inhibits microtubule depolymerization, thereby blocking the mechanism of mitosis. It is a drug molecule used in the treatment of cancers, and is injected by infusion. In oncology, paclitaxel is mainly used in lung, breast and ovarian cancer.

Side effects are those encountered with anti-cancer treatments: a drop in white blood cells, platelets and red blood cells, hair loss and inflammation of mucous membranes, particularly in the mouth. Specific side effects of paclitaxel include peripheral nerve damage, which is sometimes severe, a risk of fluid retention (ascites, pleural or pericardial effusions), skin reactions, nail damage and injection hypersensitivity reactions.

Docetaxel is an alkaloid obtained by hemisynthesis from a molecule extracted from European yew. Docetaxel is an analog of paclitaxel, with a similar structure but double the activity. It differs above all in its toxicity and antitumor efficacy. Docetaxel also stabilizes microtubules by inhibiting their depolymerization through stable binding to tubulin, resulting in blockage of mitosis. It is injected intravenously.

The use of docetaxel leads to bone marrow toxicity, to the occurrence of edema and effusion due to water retention, and to neurological disorders. That being said, it is effective for quite a large number of tumors.

Platinum salts are a class of platinum-based chemotherapy drugs. This class includes three commonly-used molecules: cisplatin, oxaliplatin and carboplatin. They are often referred to as alkylating agents, but essentially they make guanine-guanine adducts in the DNA double helix to form the DNA-platinum complex, which blocks DNA replication, transcription and repair. They also have immunosuppressive properties.

Platinum salts are potent cytotoxic agents. Thus, they cause:
nephrotoxicity, which is acute tubular necrosis that can lead to chronic renal failure;
ototoxicity, with possible permanent loss of high-frequency hearing;
vision disorders;
taste disorders;
neurotoxicity: peripheral neuropathies;
neutropenia;
myelosuppression;
frequent nausea and vomiting; and
allergic reactions.

According to the invention, the term “treatment” means an improvement, prophylaxis or reversal of a disease or disorder, or of at least one discernible symptom thereof. This is also an improvement, prophylaxis or reversal of at least one measurable physical parameter related to the treated disease or disorder, which is not necessarily perceptible to the subject. In another embodiment, the term “treatment” refers to inhibiting or slowing down the progression of a disease or disorder, either physically, for example, stabilizing a discernible symptom, physiologically, for example, stabilizing a physical parameter, or both. The term “treatment” also indicates the delay in the appearance of a disease or disorder. In certain particular embodiments of the invention, the composition of interest is administered as a preventive measure. In this context, the term “prevention” refers to a reduction in the risk of acquiring a specified disease or disorder.

More particularly, the applicant has been able to demonstrate that the composition used according to the invention is particularly effective for the prevention and/or treatment of chemo-induced nociceptive and/or neuropathic pain.

Nociceptive pain results from activation of the nociceptive system by internal or external damage to the body. In oncology, this explains more than 70% of pain syndromes. The damage may be caused by cancer and also by cancer treatments.

Neuropathic pain is the result of damage to the nervous system: compression of a nerve by the tumor, neurological toxicity of chemotherapy, sequelae of surgery, for example. It is perceived unusually: as burning, tingling, electric shocks. When the cause of neurological suffering disappears, the pain may persist, either transiently or chronically (painful sequelae). Diagnosis thereof is sometimes difficult, as this pain can occur with a time lag after nerve damage. In oncology, neuropathic pain is found alone or associated with nociceptive pain.

The different types of neuropathic effects from which patients treated with platinum derivatives or taxanes suffer are described in table 1 above.

The channels involved in nociception and mechanosensation are described in table 2 above. The applicant was able to demonstrate that oxidative stress induced by these anti-cancer agents strongly disrupts the expression of some of these channels in cultured sensory neurons. The applicant has also demonstrated that the addition of NAC, a powerful antioxidant that is perfectly well tolerated in humans, strongly limits this disruption in expression and suppresses the resulting neuropathies in a mouse model.

As shown in example 5, the applicant was able to demonstrate the existence of a synergy between NAC and vitamin B12 at a concentration of between 0.04 and 0.08% by weight of the total weight of the composition, since the latter makes it possible to lower the useful doses of NAC while retaining the same effect.

The composition used according to the invention comprises NAC at a concentration of between 10 and 90%, for example 15%, 20%, 25%, 30%, 35%, 40%, 45%, 50%, 55%, 60%, 65%, 70%, 75%, 80%, 85%, preferentially between 20 and 80%, more preferentially between 25 and 75%, for example 25% and 75%, by weight of the total weight of the composition.

The composition used according to the invention comprises vitamin B12 at a concentration of between 0.04 and 0.08%, for example 0.05%, 0.06%, 0.07%, preferentially between 0.05 and 0.07%, for example 0.06%, by weight of the total weight of the composition, taken alone or associated with a combination of vitamins B1, B2 and B3.

According to another embodiment, the composition used according to the invention comprises a combination of vitamins B1, B2 and B3, in different or equal proportions, preferentially in equal proportions, at a total concentration of between 20 and 90%, for example 15%, 20%, 25%, 30%, 35%, 40%, 45%, 50%, 55%, 60%, 65%, 70%, 75%, 80%, 85%, preferentially between 40 and 60%, for example 50%, by weight of the total weight of the composition, taken alone or associated with vitamin B12, preferentially associated with vitamin B12 at a concentration of between 0.04 and 0.08%, more preferentially between 0.05 and 0.07% by weight of the total weight of the composition.

Advantageously, according to a first embodiment, the composition used according to the invention comprises:
between 50 and 90% NAC by weight of the total weight of the composition; and
between 0.04 and 0.08% vitamin B12 by weight of the total weight of the composition.

Particularly advantageously, the composition used according to the invention comprises:
between 70 and 80% NAC by weight of the total weight of the composition; and
between 0.05 and 0.07% vitamin B12 by weight of the total weight of the composition.

Alternatively, according to another embodiment, the composition used according to the invention comprises:
between 10 and 30% NAC by weight of the total weight of the composition; and
between 20 and 90% of a combination of vitamins B1, B2 and B3 by weight of the total weight of the composition.

Advantageously, the composition used according to the invention comprises:
between 15 and 30% NAC by weight of the total weight of the composition; and
between 30 and 60% of a combination of vitamins B1, B2 and B3 by weight of the total weight of the composition.

Alternatively, according to another embodiment, the composition used according to the invention comprises:
between 10 and 90% NAC by weight of the total weight of the composition; and
between 0.04 and 0.08% vitamin B12 and between 20 and 90% of a combination of vitamins B1, B2 and B3 by weight of the total weight of the composition.

Advantageously, the composition used according to the invention comprises:
between 15 and 30% NAC by weight of the total weight of the composition; and
between 0.05 and 0.07% vitamin B12 and between 30 and 60% of a combination of vitamins B1, B2 and B3 by weight of the total weight of the composition.

Preferably, the daily dose of NAC in the composition used according to the present invention is between 200 and 1200 mg and the daily dose of vitamin B12 is between 0.16 and 1 mg, and/or the daily dose of the combination of vitamins B1, B2 and B3 is between 400 and 2400 mg, more preferentially between 280 and 1200 mg NAC, between 0.50 and 1 mg of vitamin B12 and/or between 560 and 2400 mg of the combination of vitamins B1, B2 and B3.

This daily dose is preferentially administered in 1, 2 or 3 doses (morning, midday and/or evening), for example in the form of 1, 2, 3, 4, 6, 7, 8, 9, 10, 11 or 12 tablets.

The composition used according to the invention can be administered orally, by injection or transdermally, preferentially orally.

Advantageously, the composition used according to the invention can be administered orally, in one or more identical or different formulations. It is available in any dosage form normally used for oral administration, and in particular in the form of gel capsules, tablets, capsules, soft capsules, lozenges, sachets, tubes, vials, chewing gum, pearls, emulsions, suspensions, liquids, solutions, ampoules, beverages, syrups, powders, for example contained in sachets, in particular alu-alu sachets, solids, soft gels and semi-solids.

Advantageously, the composition used according to the invention is formulated in the form of tablets, gel capsules, capsules, soft gels, semi-solids, solids, liquids, or powder.

Even more advantageously, the composition used according to the invention is formulated in the form of controlled-release microgranules in gel capsules.

Advantageously, the microgranules are coated with a semi-permeable membrane that protects the active substances from degradation and thus controls their release. This membrane thus makes it possible to mask the taste and/or odor of the substances it coats, ensuring substance stability and protecting the active ingredients from stomach acidity. It also makes it possible to control the release of the active agents.

The microgranules are preferentially in the form of microgranules of xylitol and a blend of plants, thereby enabling better assimilation by the body.

An example of the manufacturing method for these microgranules is described in example 8.

As a non-limiting example, the final weight of the gel capsule is preferentially between 200 and 600 mg. More preferentially still, it is between 300 and 500 mg. Particularly advantageously, the final weight of the gel capsule is 400 mg.

According to another embodiment, the composition used according to the invention can be administered by injection in liquid form, for example as an adjuvant in a chemotherapy solution.

According to another embodiment, the composition used according to the invention can be administered transdermally, for example in the form of a patch or medical device which is able to increase penetration through the skin to reach the bloodstream without injection, advantageously with sustained and continuous release.

Another subject of the invention is a product comprising:
NAC as first active ingredient, and
vitamin B12 at a concentration of between 0.04 and 0.08% by weight of the total weight of the composition, and/or a combination of vitamins B1, B2 and B3, as second active ingredient,
   as a combination product for use thereof simultaneously, separately or staggered over time in the prevention and/or treatment of chemo-induced nociceptive and/or neuropathic pain.

According to one embodiment, the invention relates to a product comprising:
NAC as first active ingredient,
vitamin B12 at a concentration of between 0.04 and 0.08% by weight of the total weight of the composition as second active ingredient, and
   as a combination product for use thereof simultaneously, separately or staggered over time in the prevention and/or treatment of chemo-induced nociceptive and/or neuropathic pain.

Alternatively, the invention relates to a product comprising:
NAC as first active ingredient, and
a combination of vitamins B1, B2 and B3 as second active ingredient,
   as a combination product for use thereof simultaneously, separately or staggered over time in the prevention and/or treatment of chemo-induced nociceptive and/or neuropathic pain.

According to another embodiment, the invention relates to a product comprising:
NAC as first active ingredient,
vitamin B12 at a concentration of between 0.04 and 0.08% by weight of the total weight of the composition as second active ingredient, and
a combination of vitamins B1, B2 and B3 as third active ingredient,
   as a combination product for use thereof simultaneously, separately or staggered over time in the prevention and/or treatment of chemo-induced nociceptive and/or neuropathic pain.

### EXAMPLES

### Example 1: Identification of 2 Genes for which the Change in their Expression is Common to the Different Agents Used

The inventors sought to assess how neuropathic side effects begin to take hold in the medium term (the effects on which it is most beneficial to take action).

More particularly, the inventors worked on variations in the levels of expression of the channels that are most likely to explain what is happening in this time window.

From the mechanistic perspective, it is clear that remodeling the expression of different channels depending on the agent used in chemotherapy can give rise to similar symptoms.

The aim is to identify genes for which the remodeling is similar in platinum-based and taxane-based chemotherapies.

### Materials and Methods

### 1. Primary Culture of Mouse Dorsal Root Ganglia (DRG)

Model: C57Bl6 females, 8 to 15 weeks old (sexually mature young adult mice)

Female mice were chosen to be studied because the onset of chemo-induced peripheral neuropathy is not linked to the sex of the mouse or the patient.

### 24 H Before Dissection:

### Preparation of Solutions

i. HBSS-GLUCOSE:
50 mL HBSS 10×
1.7 mL 1.5 M HEPES
10 mL 0.5 M D-Glucose
5 mL peni-streptomycin
QS to 100 mL H2O miliQ
Adjust pH to 7.5 with NaOH
QS to 500 mL H2O miliQ, Filter and bottle (store at 4° C.).
   ii. Neurobasal Medium with NGF:

For 25 mL of medium: 500 μL B27, 250 μL peni-streptomycin, 250 μL L-glutamine, 6.25 μL NGF (2000 μg/mL) and make up to 25 mL with neurobasal medium.

iii. Enzymatic Digestion:

Add 80 mg collagenase II and 200 mg dispase to 4 mL HBSS-glucose, then aliquot 200 μL per tube into 2 mL tubes and freeze.

### 12-Well Plate Coating

24 hours before dissection, a 12-well plate must be coated.

Over 2 h at room temperature, add 500 μL of PLL (poly-L-lysine) at 100 μg/mL per well; then add 500 μL of laminin at 20 μg/mL for 1 h at 37° C.

Rinse with 1× HBSS and leave to dry under fume hood overnight.

### Dissection of the Mouse

Dissection and extraction of DRGs should not exceed 1 h, to limit cell damage. The mouse is dissected ventrally, the spinal column is extracted and placed under a binocular magnifier to extract the DRGs therefrom.

Extraction takes place under “dry” conditions (not in cold PBS), and the spinal column and DRGs are regularly sprayed with cold HBSS-glucose to prevent them from drying out. After recovering between 20-30 DRGs in a 2 mL tube placed on ice, they are placed back under the binocular magnifier to cut the filaments using a scalpel (only the clusters of neurons are kept).

The DRGs are then collected in a 15 mL tube and placed on ice until ready for cell culture.

### Enzymatic Digestion of DRGs

The DRGs are rinsed twice with HBSS-glucose at room temperature.

A 2 mL tube containing the dispase and collagenase II enzyme mixture is recovered, to which 50 μL of 0.2 M CaCl₂) is added thereto.

1750 μL of HBSS-glucose is then added.

Place 1 mL of the enzymatic digestion on the DRGs and place at 37° C. for 20 minutes.

Place a further 1 mL of the enzymatic digestion on the DRGs and place at 37° C. for 20 minutes.

During enzymatic digestion, coat 3 syringes: 21 G, 22 G and 26 G with FCS (fetal calf serum).

Once enzymatic digestion is complete, rinse twice with HBSS-glucose, rinse once with 500 μL of Neurobasal medium with NGF.

Add the required amount of Neurobasal medium with NGF to the wells (200 μL/well).

Pass the DRGs through syringes from 21 G to 26 G, 6 passes for each gauge.

Then add 200 μL per well and place at 37° C. for 1 hour.

Supplement with 600 μL of Neurobasal medium with NGF. Place at 37° C.

### 2. Treatment of the Cultured DRGs

The next morning, place 1.25 μg/ml cisplatin with the other molecules to be tested.

Treatment of the DRGs for 24 hours.

Extraction of the RNA and RT-qPCR

### 3. QPCR

### RNA Extraction

The RNA is extracted using Macherey-Nagel's “NucleoSpin RNA” kit.

The RNA is then assayed using the Nanodrop and stored at −80° C.

### Reverse-Transcription

Reverse transcriptions were performed following the protocol of the “SuperScript III Reverse Transcriptase” kit from Thermo Fisher Scientific, doubling the quantities of RNA and products used in order to obtain a higher concentration of cDNA.

qPCR

The qPCRs were performed with undiluted cDNA obtained by reverse-transcription using 4.625 μL of cDNA and 5.375 μL of SyberROX PCR Mix (final 10 μL per well).

Each well was produced in duplicate.

### Analysis of the Results

When the cycle threshold (CT)>35, the gene is considered to be not expressed in the cells analyzed.

To compare our results with and without treatment, we calculated the delta between the target gene and the reference gene (36B4) for each condition (e.g. dCT_{PIEZO2}=CT_{PIEZO2}−CT_{36B4}=27.4−21.6=5.8 (control); 30.6-23.4=7.2 (cisplatin)).

We then calculated the delta delta CT of the target gene for the test condition and the control conditions (e.g. ddCT_{PIEZO2}=dCT_{PIEZO2 (cisplatin)}−dCT_{TREK1(control)}=7.2−5.8=1.4).

Finally, we determined the relative expression by calculating: 2^{\hat{ }-ddCT} (e.g. for PIEZO2:2^{\hat{ }-ddCT}=2^{\hat{ }-1.4}=0.40).

In our example, the relative expression of PIEZO2 in cells treated with cisplatin is therefore 0.40 compared to the control cells in which expression is normalized to 1.

### Microscopy

In the experiments carried out and exemplified below, a phase-contrast micrograph (not shown) is systematically taken to visualize the effect of the chemotherapy treatment and the potential neuroprotective effect of the molecules tested.

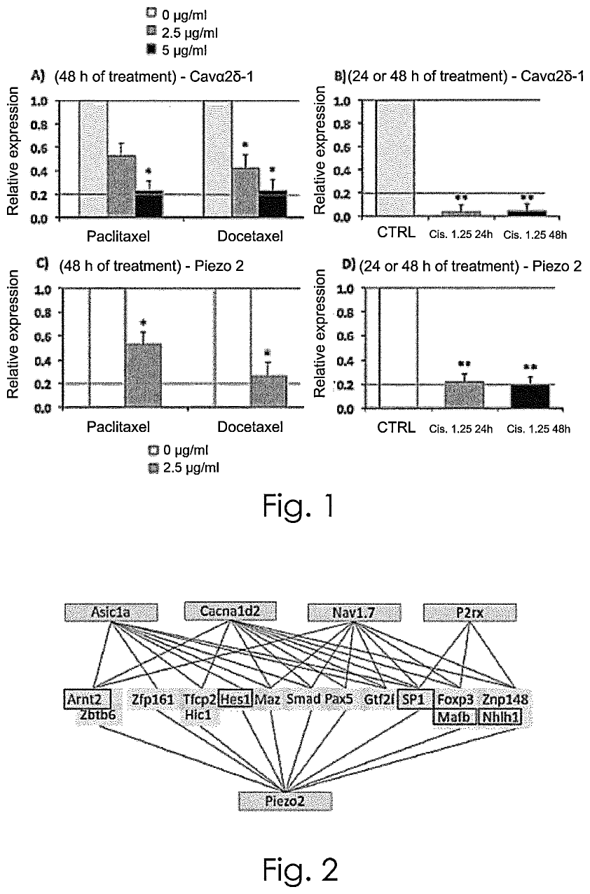

In this example on cisplatin-induced and taxane-induced gene remodeling of the ion channels expressed in mouse DRGs resulting from primary culture, according to the results obtained and shown in FIG. 1, 2 genes were more particularly identified, namely Piezo 2 and CaVα2δ-1, for which the change in expression is common to the different agents used.

The 2 genes therefore appear to exhibit a shared response to treatment by platinum derivatives and by taxanes.

The expression of Piezo 2 has therefore particularly been used as a reporter of interest for testing chemoprotective molecules.

### Example 2: Identification of Upstream Regulators

By analyzing the gene promoters for which similar changes in expression are observed, we were able to identify a number of transcription factors that may be shared regulators, as shown in FIG. 2. Of course, they must then be tested in parallel in qPCR.

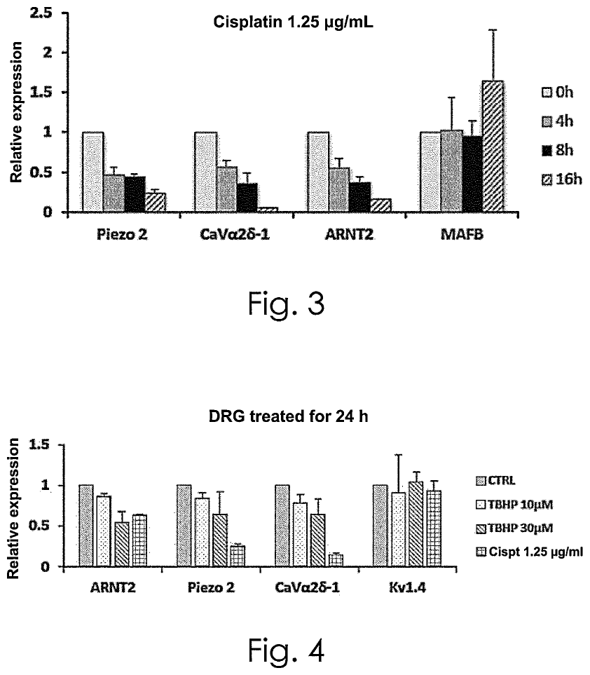

As shown in FIG. 3 relating to a treatment with cisplatin (1.25 μg/ml) in mouse DRGs resulting from primary culture, the transcription factor that has a similar pattern to Piezo and CaVα2δ-1 is ARNT2.

This is interesting because, at the protein level, it is activated both in response to oxidative stress and xenobiotics, and in turn activates genes that respond to these stresses.

This led to the hypothesis that the oxidative stress induced by the chemotherapies tested has a deleterious medium-term effect on ARNT2 expression, amplifying the toxic effect thereof in neurons.

On the other hand, as shown in FIG. 3, the effects observed for Mafb are not significant. mRNA expression was measured by qPCR and normalized with the 36B4 reference gene. The results correspond to the mean±SEM.

### Example 3: Testing the Effect of Oxidative Stress

The expression of Pizeo 2, CaVα2δ-1 and ARNT2 was measured in primary cultured neurons treated with an oxidizing agent (tert-butyl hydroperoxide, TBHP).

Expression of Kv1.4 (negative control) was unaffected.

Mouse DRGs resulting from primary culture were treated with TBHP at 10 or 30 μM or with 1.25 μg/ml cisplatin (positive control) versus control (CTRL). mRNA expression was measured by qPCR and normalized with the 36B4 reference gene. The results correspond to the mean±SEM.

This example is important because, as shown in FIG. 4, it clearly shows that there is a similar effect between the chemotherapies and an agent that causes peroxidation.

This led to testing of antioxidants on the expression of the identified reporter genes, the most obvious being NAC.

### Example 4: Testing the Effect of Different Molecules Taken Alone with Cisplatin

The effect of different molecules was tested on the expression of the same reporter genes, Pizeo 2, CaVα2δ-1 and ARNT2, using qPCR on primary cultured DRG neurons:
NAC
Beta-carotene (provitamin A)
Vitamin B12
Luteolin (flavone)

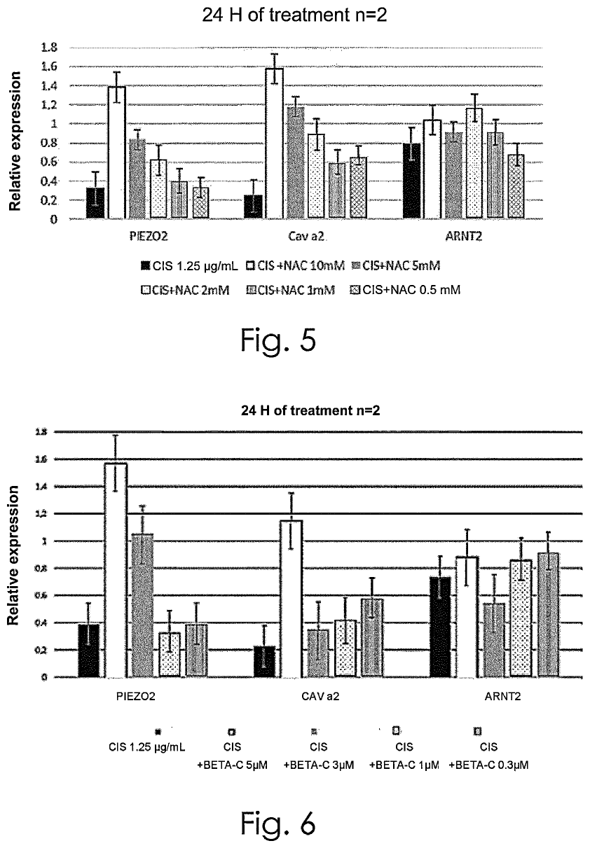

According to the results obtained, NAC appears to have a protective effect on the morphology of DRG neurons, and to allow Piezo2, CaVα2δ-1 and ARNT2 expression levels to rise ex vivo in primary cultured neurons (as shown in FIG. 5) but also in vivo in the animal exposed to a chemotherapy protocol (not shown). Finally, NAC administration appears to dramatically reduce tactile hypersensitivity (in response to mechanical stimuli induced by the Von Frey test) triggered by cisplatin and paclitaxel (not shown).

According to the results obtained and as shown in FIG. 6, beta-carotene appears to have an effect only at a 5 μM dose, and not at the lower doses tested (0.3, 1 and 3 μM). Although such a dose of 5 μM is a dose 1000 times lower than that which is useful for NAC, such a dose proves to be slightly toxic to cultured neurons (not shown).

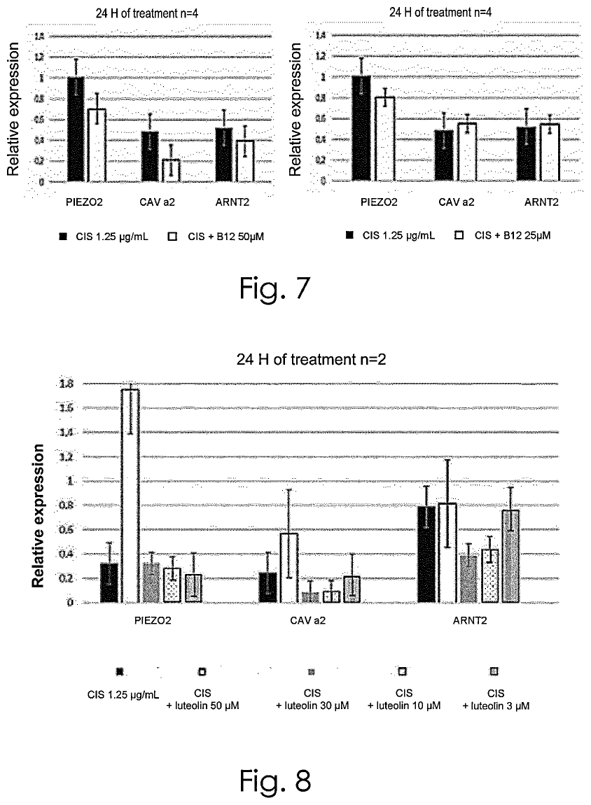

In addition, according to the results obtained and as shown in FIG. 7, vitamin B12 does not appear to have a conclusive chemoprotective effect. Vitamin B12 at a dose of 50 μM even appears to have some toxicity to neuronal morphology (not shown).

Finally, at the doses used, as shown in FIG. 8, luteolin does not appear to bring any improvement either in gene expression or in neuronal morphology at the cell or network level (not shown).

### Example 5: Testing Combinations of Molecules with Cisplatin

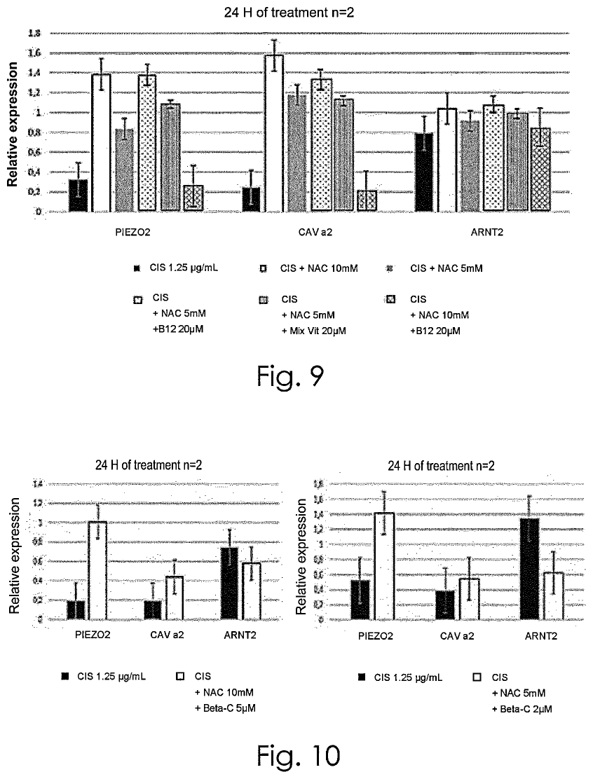

According to the results obtained, and as shown in FIG. 9, a particularly surprising synergistic effect of vitamin B12 (at a dose of 20 μM) taken in combination with NAC is observed such that, at a dose of 5 mM NAC+vitamin B12, the combination is as effective in improving the expression of genes, in particular Piezo and CaVα2δ-1, as a 10 mM dose of NAC taken alone, whereas vitamin B12 had no effect taken alone at a dose of 25 and 50 μM (see FIG. 7), and exhibits a greater effect than the same dose of NAC (5 mM) taken alone.

It is noted, however, that there appears to be a toxic effect of the combination of NAC+vitamin B12 (at a dose of 20 μM) when the concentration of NAC is doubled (10 mM).

In addition, a very good improvement in morphology and neuronal networks is observed with the combination of NAC (5 mM)+vitamin B12 (20 μM) (not shown).

Similarly, and surprisingly, a synergistic effect is also observed with the combination of NAC (5 mM)+mixture of vitamins B1, B2 and B3 in terms of improving the expression of genes, in particular Piezo, whereas the mixture of vitamins B1-B3 had no effect taken on its own.

On the other hand, by way of comparison, according to the results obtained and shown in FIG. 10, it is observed that a combination of NAC (10 mM) and beta-carotene (5 and 2 μM) is more negative than the results obtained with the same molecules taken alone (see FIGS. 5 and 6 respectively).

In addition, toxicity on neuronal cell morphology can be observed with the combination of 5 μM NAC+beta-carotene (not shown).

### Example 6: Gel Capsule A According to the Invention

According to a preferred embodiment of the invention, the composition is in the form of a gel capsule, for example of approximately 400 mg

| | % by weight of |
|---|---|
| | the total weight |
| Composition | of the composition |
| NAC | 75 |
| Vitamin B12 | 0.06 |
| Excipients (xylitol, shellac) | 24.94 |

### Example 7: Gel Capsule B According to the Invention

According to another preferred embodiment of the invention, the composition is in the form of a gel capsule, for example of approximately 400 mg

| | % by weight of |
|---|---|
| | the total weight |
| Composition | of the composition |
| NAC | 25.02 |
| Vitamin B1, B2 and B3 | 50.04 |
| (in equal proportions) | |
| Excipients (xylitol, shellac) | 24.94 |

### Example 8: Process for Producing Microgranules

### Step 1: Preparing the Shellac Solution

In a measuring cup containing a precision-weighed quantity of shellac, add a sufficient quantity of ethanol (solvent which disappears during manufacture) with stirring to prepare a 20% solution.

Stir until shellac is completely dissolved.

Step 2: Application of the NAC and Vitamin B12 and/or Vitamins B1-3 Formula

Add the specified amount of neutral microgranules into the turbine. Activate the rotation device. The NAC and vitamin B12 and/or vitamin B1-3 formula is applied by fractionation: Using the spray gun, spray some of the alcoholic shellac solution and, immediately thereafter, introduce a portion of the prescribed amount of the prepared mixture of powders of the NAC and vitamin B12 and/or vitamin B1-3 formula using a scoop.

Allow to dry between 2 sprays/applications for the required time (30 to 60 seconds) at room temperature, while maintaining rotation.

Repeat these steps until the prescribed amount of the mixture of NAC and vitamin B12 and/or vitamin B1-3 formula has been used up.

### Step 3: Screening

Screen the microgranules using a screen of appropriate diameter (1.00 to 1.18 mm). Only the microgranules passing through the screen are reintroduced into the turbine for the film-coating operation.

### Step 4: Drying

Dry the microgranules in the rotating turbine for the required time (8- to 15-hour cycle) at a temperature of between 4° and 45° C.

If necessary, particles retained on the screens are removed:
they are introduced into the turbine containing the microgranules with a sufficient amount of alcoholic shellac solution, where they undergo steps 2 and 3 again.

### Step 5: Spinning

Apply the previously prepared alcoholic shellac solution to the microgranules using the spray gun.

### Step 6: Screening

Screen the microgranules using a screen of appropriate diameter (1.18 to 1.25 mm). Only the microgranules passing through the screen are reintroduced into the turbine for the subsequent operations.

### Step 7: Drying

Dry the microgranules in the rotating turbine for the required time (8- to 15-hour cycle) at a temperature of less than 50° C.

### Step 8: Coating

Use the spray gun to apply the ready-to-use aqueous shellac dispersion to the microgranules.

### Step 9: Screening

Screen the microgranules using a screen of appropriate diameter (1.18 to 1.25 mm). Only the microgranules passing through the screen are reintroduced into the turbine for the subsequent operations.

### Step 10: Drying

Dry the microgranules in the rotating turbine for the required time (8- to 15-hour cycle) at a temperature of less than 50° C.

### Step 11: Lubrication

Introduce the specified amount of talc (0.3 to 0.4 kg) in small quantities. Stir vigorously. This facilitates the next step by reducing the static electricity of the microgranules.

### Step 12: Capsule Filling

The empty capsules are filled with microgranules up to the required fill weight.

### Step 13: Packaging

The filled capsules are packaged in blister packs and then in cardboard boxes.

1. A composition for the treatment of chemo induced nociceptive and/or neuropathic pain comprising, in a physiologically acceptable medium, N-acetylcysteine (NAC), characterized in that it further comprises vitamin B12 at a concentration of between 0.04 and 0.08% by weight of the total weight of the composition and/or a combination of vitamins B1, B2 and B3.

2. (canceled)

3. The composition according to claim 1, wherein the NAC at has a concentration of between 10 and 90% by weight of the total weight of the composition.

4. The composition according to claim 3, wherein the NAC has a concentration of between 25 and 75% by weight of the total weight of the composition.

5. The composition according to claim 1, wherein the vitamin B12 is present and has a concentration of between 0.05 and 0.07% by weight of the total weight of the composition.

6. The composition according to claim 1, wherein the combination of vitamins B1, B2 and B3 are present and have at a total concentration of between 20 and 90% by weight of the total weight of the composition.

7. The composition according to claim 6, the combination of vitamins B1, B2 and B3 has a total concentration of between 40 and 60% by weight of the total weight of the composition.

8. The composition according to claim 1, wherein vitamin B12 and the combination of vitamins B1, B2 and B3 are both present.

9. The composition according to claim 1, wherein the composition is in a form suitable for oral administration.

10. The composition according to claim 9, wherein the composition is in the form of controlled-release microgranules in gel capsules.

11. (canceled)

12. The composition according to claim 6, wherein the combination of vitamins B1, B2 and B3 has Vitamin B1, B2 and B3 present in equal proportions.

13. The composition according to claim 8, wherein the combination of vitamins B1, B2 and B3 has Vitamin B1, B2 and B3 present in equal proportions.

14. A method for treatment of chemo-induced nociceptive and/or neuropathic pain;
providing a composition comprising (i) N-acetylcysteine (NAC) and (ii) vitamin B12 at a concentration of between 0.04 and 0.08% by weight of the total weight of the composition and/or a combination of vitamins B1, B2 and B3 in a physiologically acceptable medium;
administering the composition to a user having chemo-induced nociceptive and/or neuropathic pain;
wherein administering comprises a daily dose of (i) NAC between 200 and 1200 mg and (ii) a daily dose of vitamin B12 between 0.16 and 1 mg and/or a daily dose of Vitamins B1, B2, and B3 between 400 and 2400 mg.

15. The method according to claim 14, wherein administering each daily dose comprises a plurality of partial doses until the daily dose is achieved.

16. The method according to claim 14, wherein the chemo-induced nociceptive and/or neuropathic pain of the user is a result of chemotherapy comprising platinum derivatives chosen from cisplatin, oxiplatin and carboplatin, or taxanes chosen from paclitaxel and docetaxel.

17. The method of claim 14, wherein administering comprises a simultaneous treatment of (i) N-acetylcysteine (NAC) and (ii) vitamin B12 and/or a combination of vitamins B1, B2 and B3.

18. A method for treatment of chemo-induced nociceptive and/or neuropathic pain;
providing a first composition comprising N-acetylcysteine (NAC);
providing a separate composition comprising vitamin B12 at a concentration of between 0.04 and 0.08% by weight of the total weight of the composition and/or a combination of vitamins B1, B2 and B3 in a physiologically acceptable medium;
administering the first composition and the second composition to a user having chemo-induced nociceptive and/or neuropathic pain;
wherein administering comprises a daily dose of (i) NAC between 200 and 1200 mg and (ii) a daily dose of vitamin B12 between 0.16 and 1 mg and/or a daily dose of Vitamins B1, B2, and B3 between 400 and 2400 mg.

19. The method according to claim 18, wherein administering each daily dose comprises a plurality of partial doses until the daily dose is achieved.

20. The method according to claim 18, wherein the chemo-induced nociceptive and/or neuropathic pain of the user is a result of chemotherapy comprising platinum derivatives chosen from cisplatin, oxiplatin and carboplatin, or taxanes chosen from paclitaxel and docetaxel.

21. The method of claim 18, wherein administering comprises a simultaneous, separate, or staggered treatment of the first composition and the second composition.
